(19) United States
(12) Patent Application Publication
(10) Pub. No.: US 2006/0173079 A1
(43) Pub. Date: Aug. 3, 2006

(54) Compositions and methods for improving the condition of patients suffering from copd and other diseases

(75) Inventors: Marielle P.K.J Engelen, Maastricht (NL); Nicolaas E.P Deutz, Maastricht (NL); Annemie M.W.J Schols, Maastricht (NL); Emiel F.M Wouters, Geel (BE)

Correspondence Address: KALOW & SPRINGUT LLP, 488 MADISON AVENUE, 19TH FLOOR, NEW YORK, NY, 10022, US

(73) Assignee: UNIVERSITEIT VAN MAASTRICHT, MAASTRICHT (NL)

(21) Appl. No.: 10/542,120

(22) PCT Filed: Jan. 12, 2004
(86) PCT No.: PCT/EP04/00151

§ 371 Date: Feb. 27, 2006

(30) Foreign Application Priority Data
Jan. 10, 2003 (EP) 03075179.6

(51) Int. Cl. A61K 31/198

(52) U.S. Cl. 514/566; 514/561

(57) ABSTRACT

A food supplement of therapeutic composition is provided suitable for the treatment or prophylaxis of COPD and other acute or chronic diseases in a mammal, especially a human being, comprising at least one of glutamate, other than mono sodium glutamate, and a precursor of glutamate selected from the group consisting of leucine, valine, isoleucine, and a keto acid thereof, in a daily dose for said mammal of at least 6 grams, preferably between 9 and 20 grams, of the total of said glutamate and precursor forms thereof.

### FIELD OF THE INVENTION

The present invention is in the biochemical and medical field and relates generally to nutritional and pharmaceutical compositions for improving the condition of patients suffering from Chronic Obstructive Pulmonary Disease and other acute and chronic diseases.

### BACKGROUND OF THE INVENTION

Chronic Obstructive Pulmonary Disease (COPD) represents an important health care problem in the Netherlands and abroad. COPD represents the fourth cause of death and will be the third leading cause of death worldwide in 2020, with an expected mortality of 4.7 million persons each year. Roughly 73 per 1000 persons are diagnosed as having COPD. Clinical characteristics of COPD as a rapid decline in lung function or persistently decreased lung function are observed in 20% of the general adult population in the Netherlands (1). Moreover, the total COPD related medical costs is a major burden for the Dutch health care system. The direct costs of COPD represents 1.3% of the Dutch health care budget and are expected to increase by 60% in the near future, mostly due to aging of the population (2). Currently, long-term tobacco smoking is a causal factor in more than 90% of the patients in westernized societies.

COPD is a complex clinical situation having as a common factor smoking-related, fixed airflow limitation, which does not change markedly over periods of several months of observation (3). COPD is characterized by reduced maximum expiratory flow, which is usually irreversible, and slow forced emptying of the lungs (4). Moreover, the airflow obstruction shows an abnormal rapid progressive deterioration with age. Although progression can be slowed down by medication, reversion can only be (partially) achieved through surgical interventions and transplantation. The presence of airflow obstruction in COPD is due to emphysema and/or chronic bronchitis (3). It is clinically difficult to distinguish emphysema from chronic bronchitis because of the similar symptoms of shortness of breath, cough and wheezing. In a substantial part of the patients, combinations of the characteristics ascribed to either chronic bronchitis or emphysema are present. Emphysema causes irreversible lung damage by weakening and breaking the air sacs within the lungs. As a result, elasticity of the lung tissue is lost, causing airways to collapse and obstruction of airflow to occur. Chronic bronchitis is an inflammatory disease that begins in the smaller airways within the lungs and gradually advances to larger airways. It increases mucus in the airways and increases bacterial infections in the bronchial tubes, which, in turn, impedes airflow.

The most important complaints of patients with COPD are dyspnea at exertion and in later stages also at rest, and exercise intolerance. During the last decade, research has shown that the primary lung failure is not the only factor contributing to these symptoms. Besides airflow obstruction and alveolar wall destruction, skeletal muscle dysfunction is shown to be an important determinant of dyspnea and exercise intolerance (5). This indicates the importance of considering systemic impairment in the treatment of COPD. In order to optimize the effectiveness of the COPD treatment and management, more insight is needed into the specific factors of local and systemic impairment, which underlie skeletal muscle dysfunction, as well as its interrelationship.

Peripheral skeletal muscle weakness, which is present in a substantial number of COPD patients (6, 7) and also in many other acute and chronic diseases including aging is associated with wasting of extremity fat-free mass (FFM), independent of airflow obstruction (8). In recent years, evidence revealed that the reduced exercise capacity in COPD is associated with metabolic changes. A substantial portion of patients with COPD develops lactic acidosis early in exercise and at very low work rates (9, 10). Lactic acidosis is detrimental to these patients, since it puts an additional stress on their limited ventilatory system. By enhancing the sensation of dyspnea, it may possibly contribute to their decreased exercise capacity.

Recently, evidence became available that the accelerated lactate response to exercise in COPD patients correlates with intrinsic abnormalities in metabolism of the peripheral skeletal muscle (11), as illustrated by the inverse relationship between the steepness of the lactate increase and the activity of muscle oxidative enzymes. A relative shift from oxidative to glycolytic capacity in peripheral skeletal muscle is a key finding in COPD: a decrease in the proportion of the slow-twitch type 1 fibers corresponded with a relative increase in fast-twitch type 2b/x fibers (12-14). In line with these morphological changes, reduced values were found for enzymes involved in the tricarboxylic acid cycle (citrate synthase) and in β-oxidation of fatty acids (hydroxyacyl CoA dehydrogenase) (11, 15).

The functional consequences in stable COPD patients were reflected in a marked increase in muscle Pi/PCr ratio and intracellular acidosis at the end of exercise and a slow PCr resynthesis rate, as assessed by ³¹P-Nuclear Magnetic Resonance techniques (16, 17). Moreover, alterations in adenine nucleotide metabolism and increased levels of muscular inosine mono-phosphate (IMP) are already present in COPD patients at rest (13, 18), the latter being most pronounced those with emphysema.

Consistent results in muscle amino acid profile were found in COPD patients with respect to the amino acid glutamate (GLU). In several studies, severely reduced levels for muscle GLU were found in COPD at rest (19-21). Depleted GLU levels were present in different muscle groups such as quadriceps femoris muscle and tibialis anterior muscle (19, 20). Moreover, depleted muscle GLU levels were present in all COPD patients, independent of the severity of airflow obstruction, but to a greater extent in those with emphysema (20). GLU, which comprises ˜10% of all amino acids in natural proteins, is one of the amino acids in highest concentration in the free amino acid pool in skeletal muscle but is present at a low concentration in plasma. GLU is one of the most important non-essential amino acids and takes part in numerous important metabolic processes at rest and during exercise.

First of all, GLU is an important precursor for the first and rate-limiting step in the synthesis of glutathione (GSH), which is one of the most important antioxidants in muscle. The antioxidant status determines its susceptibility to oxidative stress, which may induce muscle damage via the formation of free oxygen radicals. Unless cysteine, glycine or the corresponding enzymes become limiting, GSH level is determined by GLU concentration. A recent study in 13 emphysema patients and 25 healthy control subjects revealed reduced muscle GLU and GSH levels in the patient group (20). Moreover, muscle GLU was highly associated with GSH in both patients and controls. Oxygen desaturation is frequently present in emphysema patients during activities of daily living (e.g. meals, exercise) (22-24). An adequate level of antioxidants is of particular importance in these conditions, as intermittent hypoxia is known to increase oxidative stress (25). Therefore, the presence of increased oxidative stress in combination with reduced muscle GSH levels may result in an antioxidant to oxidant imbalance and in this way induce muscle damage in patients with emphysema.

Secondly, GLU plays a role in preserving high-energy phosphates in muscle through different metabolic mechanisms at rest and during exercise. GLU is involved in anaerobic ATP formation by enhancing substrate phosphorylation during ischemic and hypoxic conditions (26). These conditions have been shown to increase intracellular GLU degradation in heart tissue and mitochondria. Furthermore, GLU has a role in the establishment and maintenance of a high concentration of tricarboxylic acid cycle intermediates during short-term exercise (27, 28), which is achieved via the alanine aminotransferase reaction (pyruvate+GLU→alanine+α-ketoglutarate) and at the cost of GLU.

Moreover, this reaction can shunt the pyruvate accumulated during exercise towards alanine instead of lactate, and thus, thirdly, suggesting a possible role of the intracellular GLU level in the lactate response to exercise. In line with this hypothesis, early lactic acidosis during exercise in patients with COPD was indeed associated with a reduction in muscle GLU (29). This suggests that changes in muscle GLU level may also contribute to the accelerated lactate response to exercise in these patents. In addition to the reduced baseline GLU levels, low intensity exercise resulted in a further reduction in muscle GLU status (21).

A recent study in young, healthy men (mean age 26.7 y) revealed that plasma glutamate, both at rest and during exercise can be successfully elevated by the administration of mono sodium glutamate (MSG) (30, 41). Providing glutamate in excess resulted in an increase in muscle GLU concentration (41) and a further evaluation of plasma glutamate as well as aspartate during exercise compared with rest. It was further suggested that increased glutamate availability during exercise alters its distribution in transamination reactions within active muscle, which results in elevated alanine and decreased ammonia levels.

GLU in muscle is derived intracellularly by net protein degradation. Furthermore, the essential branched-chain amino acids (BCAAs) leucine, valine and isoleucine are important precursors in the formation of GLU. BCAA derived from net protein breakdown and by uptake into the muscle pool, undergo transamination to yield branched-chain keto acid and GLU. BCAA transaminase activity is high in human skeletal muscle. In plasma of COPD patients, consistently reduced levels have been found for the BCAAs as compared with healthy age-matched controls (30-33). Recently, we found that the reduced BCAA level in plasma of COPD patients was fully caused by a reduced level of leucine, but not of valine or isoleucine (33). Since no significant change was found in skeletal muscle leucine level, ratio muscle to plasma leucine was increased, indicating that specific disturbances in leucine metabolism are present in these patients. It is therefore possible that an altered BCAA (and particularly that of leucine) metabolism may contribute to the reduced GLU levels in peripheral skeletal muscle of patients with emphysema.

GLU is found both in the free form and bound in protein in virtually all protein-containing food products. However, GLU in food is especially known from its salt, monosodium glutamate (MSG) that is often used in or on a variety of foods like on meat, fish, poultry and many vegetables, and in sauces, soups and marinades to enhance flavour. MSG is formed after industrial fermentation of starch, sugar beets, sugar cane or molasses. The total average daily intake of MSG is estimated to be 0.3-1.0 g in industrialized countries, depending on the MSG content in food and the individual taste preference (34).

There has been concern about the addition of MSG to food, since several side effects have been reported after the MSG ingestion (35). A mixture of symptoms like headache, nausea, burning sensation in the back of the neck, forearms and chest, chest pain and facial pressure were described as the Chinese restaurant syndrome in relation to the frequent use of MSG in the Chinese kitchen. Since than, many animal and human studies have been performed to evaluate possible side effects of MSG ingestion (36-39). Furthermore, organisations like the Scientific Committee for Food of the Commission of the European Communities (SCF) and the Joint FAO/WHO Expert Committee on Food Additives (JECFA) have evaluated the safety of glutamate and allocated an “acceptable daily intake (ADI) not specified” to the natural glutamate and its monosodium, potassium, calcium, and ammonium salts because human studies failed to confirm the involvement of MSG in any kind of adverse effect (36). The conclusions of the Federation of American Societies for Experimental Biology (FASEB) and the Food and Drug Administration (FDA) do not discount the existence of a sensitive subpopulation but otherwise concurred with the safety evaluation of the JECFA and the SCF. Thus, the possibility to develop specific symptoms after MSG ingestion cannot be rejected.

As far as the inventors are aware, no therapeutic, prophylactic or other remedy has been proposed so far to ultimately restore or at least increase the GLU level in skeletal muscles of patients suffering from COPD and other acute and chronic diseases including aging despite the studies mentioned above.

It is therefore an object of the present Invention to relieve the condition of patients suffering from COPD and other acute and chronic diseases including aging by providing glutamate, other than mono sodium glutamate, or one or more precursors of glutamate (i.e. BCMs: (leucine, valine, isoleucine; and its keto acids) in a suitable form for administration in order to increase and/or normalize the reduced GLU status in skeletal muscle of these patients.

Based on the same principle, it is another object of the invention to restore or increase glutamate availability in the body and especially muscles of healthy people without the side effects of increasing the glutamate level according to the prior art methods, in particular by employing mono sodium glutamate.

### SUMMARY OF THE INVENTION

In accordance with the present invention a composition is provided suitable for the treatment or prophylaxis of COPD and other acute or chronic diseases in a mammal, especially a human being, comprising at least one of glutamate, other than mono sodium glutamate, and a precursor of glutamate selected from the group consisting of leucine, valine, isoleucine, and a keto acid thereof, in a daily dose for said mammal of at least 6 grams, of the total of said glutamate and precursor forms thereof. In a preferred embodiment the amount of said glutamate or said precursor of glutamate is in a range of between 9 and 20 grams of the total of said glutamate and precursor forms thereof, which Is approximately in the range of 0.12 to 0.27 g/kg body weight.

The compositions according to the invention are also suitable to restore or increase glutamate availability in the body and especially in the muscles of healthy people, for example during or after exercise, such as sports.

The compositions according to the invention are preferably in the form of a dietary food supplement where the amount of said glutamate or said precursor form thereof is preferably subdivided in dosages of up to 3 grams, for regular administration to achieve continuously increasing glutamate level.

In another preferred embodiment of the present invention the composition is a pharmaceutical composition where the amount of said glutamate or said precursor form thereof is preferably subdivided in unit dosages of up to 3 grams, for regular administration to achieve continuously increasing glutamate level, the pharmaceutical composition further comprising a pharmaceutical acceptable carrier.

In another preferred embodiment of the invention there is provided the use of at least one of glutamate, other than mono sodium glutamate, and a precursor of glutamate selected from the group consisting of leucine, valine, isoleucine, and a keto acid thereof, in the preparation of a medicament for the treatment or prophylaxis of COPD and other acute or chronic diseases including aging in a mammal, especially a human being, wherein the medicament is formulated in a unit dose form to achieve a daily dose of at least 6 grams, which is approximately equivalent to at least 0.8 g/kg body weight, preferably in a range of between 9 and 20 grams of the active ingredient of the medicament.

In still another preferred embodiment of the invention there is provided the use of at least one of glutamate, other than mono sodium glutamate, and a precursor of glutamate selected from the group consisting of leucine, valine, isoleucine, and a keto acid thereof, to increase the glutamate level in the body and especially the muscles of individuals. A preferred use includes the use in healthy people, for example in the form a food supplement or prophylactic preparation, to restore or increase the glutamate level during or after exercise.

The food supplement of pharmaceutical composition is preferably formulated for oral or parenteral administration. In a preferred embodiment of the invention the composition is formulated to achieve a continuously increasing glutamate level.

The pharmaceutical composition may additionally contain one or more substances selected from the group of stimulants, hormones, analogues of such hormones, phyto-hormones, analogues of such phyto-hormones, and anti-oxidants.

In another aspect of the invention a method is provided of preventing or treating COPD and other acute or chronic diseases including aging in a mammal, in particular a human, which comprises administering to said mammal a therapeutically effective amount of at least one of glutamate, other than mono sodium glutamate, and a precursor of glutamate selected from the group consisting of leucine, valine, isoleucine, and a keto acid thereof.

These and other aspects of the invention will be discussed in more detail below.

### BRIEF DESCRIPTION OF THE DRAWINGS

FIG. 1: Summary of pilot studies: Evaluation of plasma glutamate concentration after ingestion of 69.4 mg GLU/kg BW every 30 min (GLU2), 34.7 mg GLU/kg BW every 10 min (GLU3), 69.4 mg GLU/kg BW every 20 min (GLU4) and 30 mg GLU/kg BW every 20 min (GLU5).

FIG. 2: Mean plasma GLU concentration of 4 subjects after continuous ingestion of 30 mg GLU/kg body weight every 20 min. A steady state in GLU concentration was reached within 2 hours after start of ingestion.

FIG. 3: Mean plasma GLU concentration of 8 COPD patients and 8 healthy control subjects after continuous ingestion of 30 mg GLU/kg body weight every 20 min. 120 minutes after start of GLU ingestion, plasma GLU level was significantly increased in both groups compared to baseline values. However, the rise in GLU concentration was lower in the COPD group than in the control group.

FIG. 4: When adding a carbohydrate protein meal to the GLU ingestion in healthy young volunteers, plasma GLU concentration also significantly increased to steady state values

FIG. 5: Besides GLU concentration also whole body GLU plasma appearance reached steady state values within 1.5 hours after start of GLU ingestion. Ingestion of glutamate was started just after 90 min.

FIG. 6: Whole body phenylalanine (PHE) turnover gives a reflection of whole body protein breakdown. There is a gradual decrease in protein breakdown after start of GLU ingestion.

FIG. 7: Whole body 3-methylhistidine (3MH) turnover is a marker of myofibrillar muscle breakdown. In less than one hour after GLU ingestion, a reduction in myofibrillar protein breakdown was present.

FIG. 8: Whole body rate of appearance of 3-methylhistidine in plasma is presented of the COPD group when ingesting GLU or water before and during cycle exercise. Ingestion of glutamate abolished the increase in 3 methylhistidine rate of appearance as observed when ingesting water. In fact during the first 10 minutes of exercise, a reduction in myofibrillar protein breakdown was observed when ingesting glutamate.

FIG. 9: Absolute change in urea concentration from baseline values in the control (FIG. 9a) and COPD group (FIG. 9b). Compared to baseline values, plasma urea decreased immediately after GLU intake but increased after GLN ingestion in both the healthy control and COPD group. This reduction in plasma urea level during GLU intake remained until the end of the experiment. The reduced urea level during GLU intake reflects protein anabolism.

FIG. 10: When adding a carbohydrate protein meal to the GLU ingestion in healthy young volunteers, plasma urea concentration also decreased after intake.

FIG. 11: Whole body leucine rate of appearance gives a reflection of whole body leucine turnover and is the sum of leucine turnover coming from protein breakdown and from other (non-protein) sources. Glutamate ingestion induced a reduction in whole body leucine turnover not related to protein turnover in both COPD and control subjects.

FIG. 12: Whole body leucine rate of appearance of the COPD group is presented when ingesting 30 mg GLU/kg body weight every 20 min or the same amount of water. The subjects were measured before and during a submaximal constant work rate cycle test of 20 minutes (at 50% of their maximal workload previously achieved during an incremental exercise test). Glutamate ingestion diminished the increase in whole body leucine turnover during exercise.

FIG. 13: Mean plasma lactate concentration is presented during and after the 2 exercise bouts in the healthy control group. Glutamate ingestion resulted in a lower increase in plasma lactate concentration during exercise than when ingesting water.

FIG. 14: Overview of all complaints including those often attributed as Chinese restaurant syndrome. The percentage of people who reported symptoms to a mild degree after GLU ingestion is presented.

FIG. 15: Overview of symptoms of the Chinese restaurant syndrome until 2 hours after ingestion of GLU. The percentage of people who reported symptoms to a mild degree is presented.

### DETAILED DESCRIPTION OF THE INVENTION

As used herein, the term “glutamate” generally refers to L-glutamic acid units in peptides at a level higher than present in naturally-occurring proteins such as vegetable, animal or diary protein (usually containing less than 10 g L-glutamic acid/100 g protein), or L-glutamic acid in the free form solved in water resulting in a solution, or added to food as dry L-glutamic acid in the free form, unless stated otherwise.

The reduced GLU level, which has consistently been found in skeletal muscle of patients with COPD, and the possible negative effect on glutathione, protein and energy metabolism (see above) indicate the importance of normalizing GLU level in these patients. GLU level in skeletal muscle can theoretically be enhanced via intravenous infusion or oral supplementation of the amino acid GLU as a component of proteins or peptides or in a free form. However, as disturbances in skeletal GLU metabolism has been observed in a very large group of COPD patients, oral supplementation as a therapeutic way to modulate GLU metabolism is preferable.

However, it is generally thought that when ingesting a low dose of GLU, GLU will largely be extracted by the splanchnic area for oxidation and transamination, resulting in only a very small increase in systemic plasma GLU. When this is the case, GLU concentration will not rise significantly in skeletal muscle. A recent study however concluded that it is actually possible to increase the GLU concentration in muscle in healthy volunteers once GLU concentration in plasma is enhanced (40).

So, the first important issue that had to be covered was the preparation of a GLU enriched drink that was able to increase plasma GLU concentration. In order to study GLU turnover (synthesis/breakdown) and related metabolism using stable isotope methodology, it was necessary to develop a GLU enriched drink protocol that was able to increase plasma GLU concentration to a steady state level. When using a continuous infusion protocol of stable isotopes, plasma glutamate concentrations have to be in steady state condition before metabolic data can be obtained.

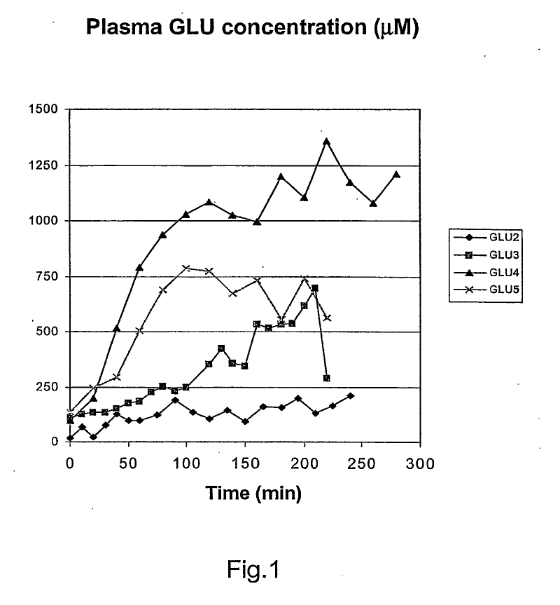

First of all, extensive literature research was done on the metabolic routes in which GLU is involved, and on the possible (often presumed) side effects of ingestion of monosodium glutamate (MSG), the sodium salt of glutamate which has mostly been used in the literature. We concluded that there exists a sensitive group of people being intolerant for MSG. Subsequently, we performed several pilot studies (summary see FIG. 1) in order to obtain the optimal dose of glutamate. Pilot studies included continuous ingestion of a glutamate enriched drink and evaluation of the concentration of glutamate and related amino acids in plasma.

In the first pilot study, we gave a 3.6% MSG solution of 80.3 mg MSG/kg body weight every 30 minutes to healthy volunteers (same dose as used by Graham (40) but given continuously and not as a bolus) to get some information about the taste and tolerance of the drink. The taste was very salty but the tolerance was well.

In the 2^{nd} pilot study, we decided to use the pure form of glutamate to avoid the large sodium content. To obtain the same amount of glutamate as in MSG, 69.4 mg glutamate/kg body weight was provided to healthy volunteers every 30 minutes (a 2.4% solution). Blood samples were taken to evaluate plasma glutamate level. The data revealed that 30 min intervals were too long to reach a steady state in plasma glutamate level.

A 3^{rd} and 4^{th} pilot study was performed using the same total amount of glutamate ingested (69.4 mg glutamate/kg body weight) but the time intervals were 10 and 20 min, respectively. The results of the 4th pilot study were promising although the total amount of ingested glutamate was quite high (614.5 mg/kg body weight).

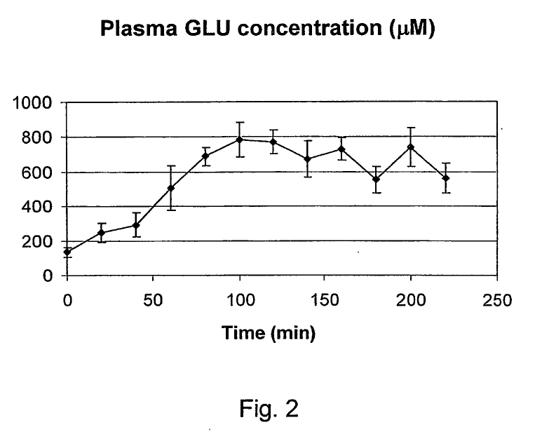

In the 5^{th} pilot study, we reduced the total GLU intake to a total amount of 300 mg glutamate/kg body weight. This resulted in an increase in the plasma glutamate concentration of about 500% and the steady state was reached within 120 minutes (FIG. 2). This protocol was used in further studies.

Based on these results it was thought that ingestion of GLU may be an efficient substrate to restore the decreased muscle GLU levels in COPD. However, in the study by Graham et al. (40), a high (bolus) dose of monosodium glutamate (MSG) (the sodium salt of glutamate) was used, and several subjects experienced transient headaches related to the ‘Chinese restaurant syndrome’ (CRS). This is a group of symptoms (such as headache, pain on the chest, nausea, dyspnea), which has occasionally been reported in subjects after eating a Chinese meal. The average daily intake of MSG is estimated to be 0.3-1.0 g in industrialized countries, but can be higher occasionally (double), depending on the MSG content of individual food items and an individual's taste preferences.

In accordance with the present invention it has now been found that these side effects do not occur when applying glutamate other than MSG, while the efficiency remained at the same level as with MSG.

The compositions for the treatment or prophylaxis of COPD and other acute or chronic diseases including aging according to the present invention are suitably administered to the mammal in the form of a food supplement or pharmaceutical composition. The administration may be preferably by way of oral or parenteral administration.

When the composition is in the form of a food (or nutritional) supplement, the latter comprises for example a palatable base which acts as a vehicle for administering the composition to an individual and which can mask any unpleasant taste or texture of the composition. The food supplement may contain any one or several nutrients including drugs, vitamins, herbs, hormones, enzymes and/or other nutrients. The nutritional supplement may contain plural parts, where each of the plural parts is chronologically appropriate for its scheduled time of consumption. For the desired or preferred amounts of the compositions according to the present invention to be dosed to individuals, for example on a daily basis, reference is made to the dosages mentioned below in connection with the pharmaceutical compositions. Similar amounts of the active ingredients (i.e. glutamate and/or its precursor forms) are applicable in the food supplement compositions of the present invention.

When the composition is in the form of a pharmaceutical composition, it can be administered in conventional form for oral administration, e.g. as tablets, lozenges, dragees and capsules. However, in certain cases it may be preferred to formulate the composition as an oral liquid preparation such as a syrup, a nasal spray, or a suppository. The medicament can also be administered parenterally, e.g. by intramuscular or subcutaneous injection, using formulations in which the medicament is employed in a saline or other pharmaceutically acceptable, injectable composition.

An amount effective to treat the disorder hereinbefore described depends on the usual factors such as the nature and severity of the disorder being treated, the weight of the mammal, the specific compound(s) of choice, glutamate itself or one of the precursor forms thereof, and considerations and preferences of the prescriber. The amount of active ingredient(s) to be administered usually will be in the range of up to 3 grams per dose. However, a unit dose will normally contain 2 to 3 grams. Unit doses will normally be administered once or more than once per day, for example 3, 4, 5 or 6 times a day, more usually 4 to 6 times a day, such that the total daily dose is normally in the range, for a 75 kg adult, of 9-20 grams, that is in the range of approximately 0.12 to 0.27 g/kg/day.

It is greatly preferred that the glutamate and/or a precursor form and/or a pharmaceutically acceptable salt thereof according to the present invention is administered in the form of a unit-dose composition, such as a unit dose oral, such as sub-lingual, rectal, topical or parenteral (especially intravenous) composition.

Such compositions are prepared by admixture and are suitably adapted for oral or parenteral administration, and as such may be in the form of tablets, capsules, oral liquid preparations, powders, granules, lozenges, reconstitutable powders, injectable and infusable solutions or suspensions or suppositories. Orally administrable compositions are preferred, in particular shaped oral compositions, since they are more convenient for general use. The preparation of such compositions is well known to people skilled in the art and can be optimized in a routine way without exerting inventive skill and without undue experimentation.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well-known methods in the art.

Suitable fillers for use include, mannitol and other similar agents. Suitable disintegrants include starch derivatives such as sodium starch glycollate. Suitable lubricants include, for example, magnesium stearate.

These solid oral compositions may be prepared by conventional methods of blending, filling, tabletting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

Oral formulations further include controlled release formulations, which may also be useful in the practice of this invention. The controlled release formulation may be designed to give an initial high dose of the active material and then a steady dose over an extended period of time, or a slow build up to the desired dose rate, or variations of these procedures. Controlled release formulations also include conventional sustained release formulations, for example tablets or granules having an enteric coating.

Nasal spray compositions are also a useful way of administering the pharmaceutical preparations of this invention to patients such as children for whom compliance is difficult. Such formulations are generally aqueous and are packaged in a nasal spray applicator, which delivers a fine spray of the composition to the nasal passages.

Suppositories are also a traditionally good way of administering drugs to children and can be used for the purposes of this invention. Typical bases for formulating suppositories include water-soluble diluents such as polyalkylene glycols and fats, e.g. cocoa oil and polyglycol ester or mixtures of such materials.

For parenteral administration, fluid unit dose forms are prepared containing the compound and a sterile vehicle. The compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle.

Parenteral suspensions are prepared in substantially the same manner except that the compound is suspended in the vehicle instead of being dissolved and sterilised usually by exposure to ethylene oxide before suspending in the sterile vehicle. Advantageously, a surfactant or wetting agent is included in the composition to facilitate uniform distribution of the compound of the invention.

As is common practice, the compositions will usually be accompanied by written or printed directions for use in the medical treatment concerned.

In the treatment of COPD and other patients in accordance with the invention, glutamate and/or a precursor form can be used alone or together with other active materials. The latter materials are preferably chosen such that either their activity is enhanced, preferably in a synergistic way, or undesired side-effects are suppressed by the glutamate and/or one of its precursor forms. For example, glutamate and/or one of its precursor forms which can be used in conjunction with the medicament additionally contains one or more substances selected from the group of stimulants, hormones, analogues of such hormones, phyto-hormones, analogues of such phyto-hormones like phyto estrogen, and anti-oxidants like phyto vitamins c and e, flavonoids.

Preliminary investigations show the following suitable dose rates: up to 3 g oral or sublingual dosage (PO) per 20 minutes during at least 2 hours. May take up to 18 g PO if needed.

In all the pilot studies, the patients and the healthy control subjects in the age ranging from 20 to 80 years were run through a protocol to determine feasibility. The above-mentioned protocol did not induce any adverse complaints in any of the study subjects. The study subjects were tolerating the given oral doses well and were able to complete the protocol.

The compositions according to the present invention are useful for the treatment of individuals suffering from COPD or other acute and chronic diseases such as chronic heart failure, renal failure, cancer, sepsis, acute liver failure, acute pancreatitis and also aging, to relieve their condition and/or to increase and/or normalize the reduced GLU status in skeletal muscle of these individuals (42-44) without the side effects which are known to occur when similar amounts of MSG would have been used. The determined diseases are to be understood broadly and include also acute metabolic stress conditions such as surgical trauma and injury, which are also characterized by a reduction in muscle GLU concentration (45, 46). The compositions of the present invention are also useful for healthy people to restore or increase glutamate levels in the body and especially the muscles, in particular during or after exercise such as sports. It has been shown that physical exercise in healthy people and also in diseases such as COPD is associated with a reduction in skeletal muscle GLU concentration (21, 27, 47).

Although the invention has been described primarily as a therapy for adults, it can also be used for children, if necessary, although dosage rates may be different in the case of children. Adaptation and optimization of dosages can be readily achieved by skilled persons without undue experimentation.

The following non-limiting examples illustrate the invention.

Materials and Methods

The metabolic consequences of GLU ingestion using above-mentioned protocol were examined in healthy young (mean age 25 y) and elderly subjects (mean age 65 y), and in patients with moderate to severe COPD according to the American Thoracic Society guidelines (3) (forced expiratory airflow obstruction in 1 second (FEV₁) less than 70% of predicted (mean FEV₁: 47.5±4.6% of predicted, mean age 65 y). A continuous dose of GLU was given because a steady state condition is necessary to estimate the effect of the GLU ingestion on protein and related metabolism using the stable isotope technique. The results of those studies are described below:

Plasma GLU Increase After GLU Ingestion

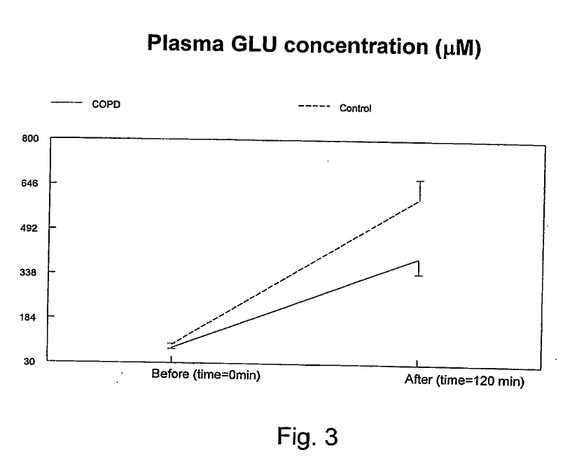

A continuous ingestion of 30 mg GLU/kg body weight every 20 min (2.4% solution) in young volunteers resulted in a plasma glutamate concentration of about 500% and the steady state was reached within 120 minutes (FIG. 2). This indicates that GLU ingestion according to this protocol actually leads to a rapid and significant increase in GLU concentration in plasma. This was confirmed in a later study in COPD patients and healthy age-matched (elderly) subjects. GLU ingestion resulted in a significant increase in plasma GLU level in both groups. Interestingly, the level of GLU increase was significantly lower in the COPD group than in the control group (FIG. 3), suggesting that glutamate uptake (in splanchnic area and/or periphery) is enhanced in COPD.

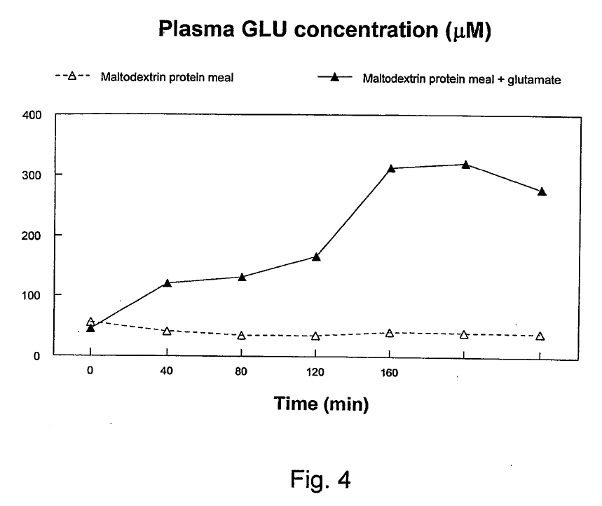

To see whether the findings observed after free GLU ingestion were also present when adding a carbohydrate (maltodextrin) whey protein meal (carbohydrate:protein:glutamate=5:2:5) to the glutamate, a pilot study in young volunteers was performed (FIG. 4). Data indicate that plasma GLU concentration also significantly increased to steady state values when GLU was added to a carbohydrate protein meal.

GLU Appearance in Plasma and Splanchnic GLU Extraction

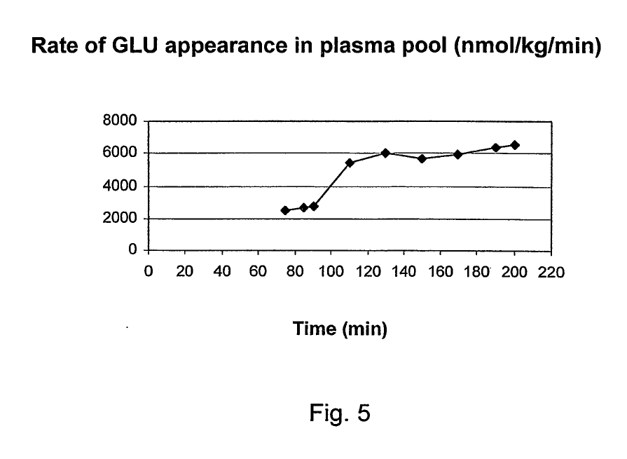

In order to examine whether this GLU increase in plasma is actually due to the increase appearance of GLU in plasma related to GLU ingestion, GLU appearance in plasma was measured before and during GLU supplementation in the young subjects using stable isotope methodology. GLU appearance in the plasma pool quickly increased after start of ingestion and reached a steady state within 1.5 hours (FIG. 5).

It was calculated that GLU splanchnic extraction will be between 41 and 66% after GLU ingestion assuming either an inhibition of endogenous GLU release to zero or no inhibition. These results suggest that between 59% and 34% of the ingested GW actually entered the systemic circulation (plasma pool). This finding is remarkable since until yet only data are available showing a much larger extraction of GLU in the splanchnic area.

Research by Matthews and colleagues, who used the GLU tracer both intravenously and enterally to measure intestinal GLU metabolism, showed that enterally infused GLU was extracted to a large extent in the intestine (88%) (48). The major fate of GLU extraction in the intestine is oxidation, although a small increase in [¹⁵N]-enrichment was also present in other amino acids (ie glutamine). Further studies on GLU metabolism in the pig's intestine was done by the group of Reeds (49). Their results strengthen the conclusions that has been made by Matthews et al. that the GLU given enterally is the major substrate for the energy production in the intestine and for that reason the major part of enteral GLU ingestion will not appear in the systemic circulation.

However, both Matthew et al. and Reeds et al. have used much smaller GLU amounts of enteral GLU substrate in the postabsorptive state than used in our recent pilot studies. In this state, the intestine is very sensitive to all nutrients and these small amounts of labelled GLU will disappear immediately after reaching the lumen of the intestine. In contrast, in the study by Stegink et al (50) and Ghezzi et al (51), who used various concentrations of GLU in the form of monosodium glutamate in healthy volunteers, plasma GLU concentration increased proportionally to the given dose. Their findings are in line with ours suggesting that after administration of larger doses of MSG or GLU, the metabolic capacity of the intestine for GLU has reached its maximum and the excess GLU enters the systemic circulation.

Whole Body and Muscle Protein Metabolism

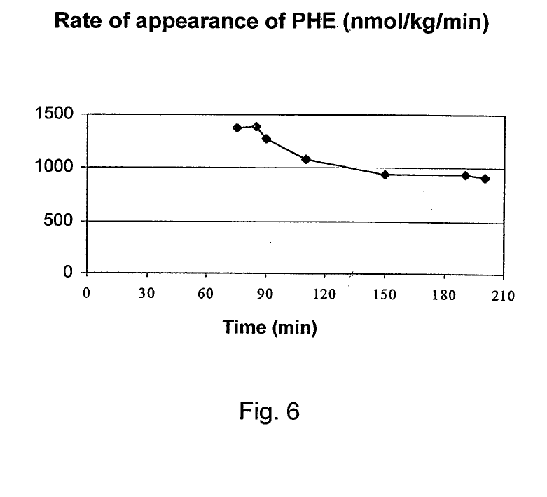

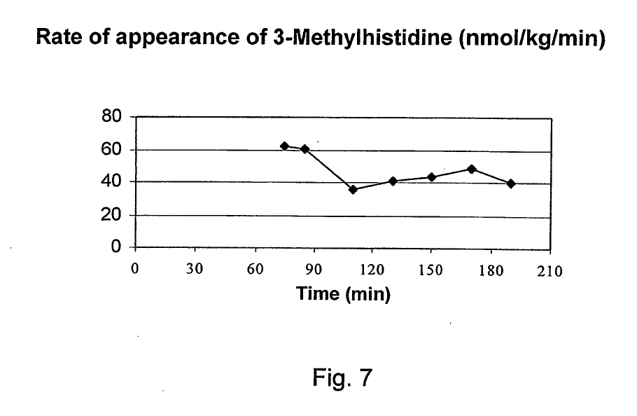

In the young healthy volunteers, whole body protein and 3-methylhistidine turnover were measured during glutamate supplementation using stable isotope methodology. Whole body protein breakdown rate significantly decreased during GLU supplementation (FIG. 6). In order to elucidate the contribution of muscle to whole body protein metabolism, the rate of myofibrillar protein breakdown (3-methylhistidine turnover) was simultaneously measured. It appeared that also 3-methylhistidine breakdown rate decreased during GLU ingestion (FIG. 7), suggesting an anabolic effect of GLU not only on whole body level but also on muscle level.

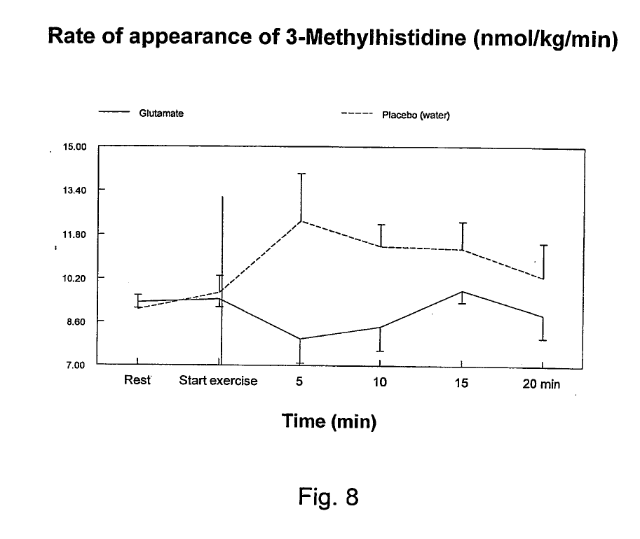

Submaximal cycle exercise (at 50% of their maximal workload previously achieved during an incremental exercise test) during 20 minutes resulted in an increase in 3-methylhistidine rate of appearance in plasma of the COPD patients, suggesting that exercise increases myofibrillar (muscle) protein breakdown in COPD. Ingestion of glutamate diminished 3-methylhistidine rate of appearance (FIG. 8), indicating that glutamate reduces myofibrillar (muscle) protein breakdown during exercise in COPD.

Plasma Urea Concentration

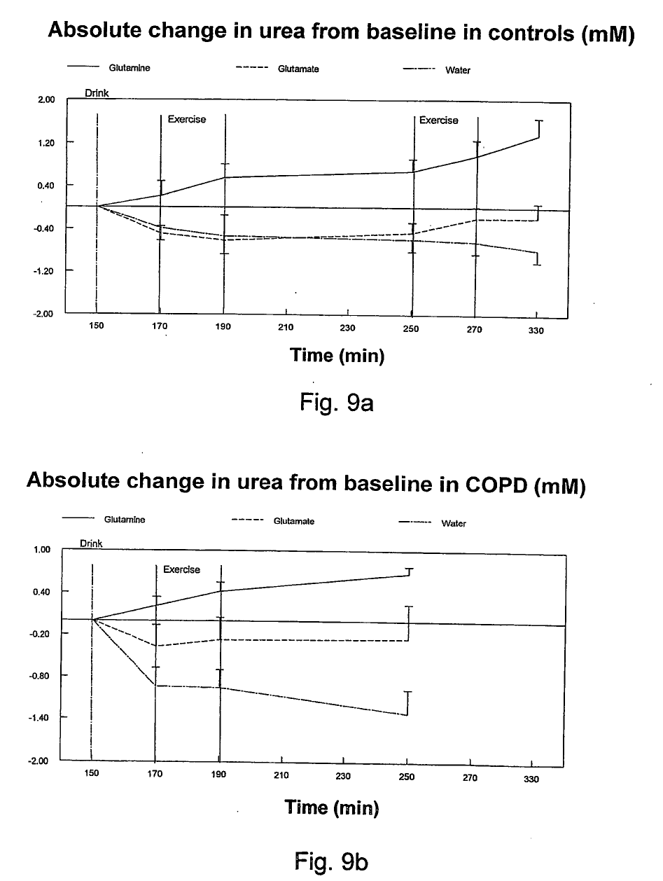

Urea concentration was measured in COPD subjects and age-matched control subjects after ingesting a 2.4% solution of 30.0 mg GLU/kg body weight, an isomolar amount of a control drink (29.8 mg glutamine/kg BW) or the same amount of water every 20 min for the following 4 hours. The 3 test drinks were studied on different days and in a randomized order using a similar study protocol. Compared to baseline values, plasma urea decreased immediately after GLU intake but increased after GLN ingestion in both the healthy control and COPD group (FIGS. 9a and b). This reduction in plasma urea level during GLU intake was present at rest, during exercise until at least 1 hour in recovery. The reduced urea level indicated that the ingested nitrogen remained in the body and is not wasted out like it is the case for glutamine. This is in line with the previous observation that glutamate induces protein anabolism in COPD and healthy controls.

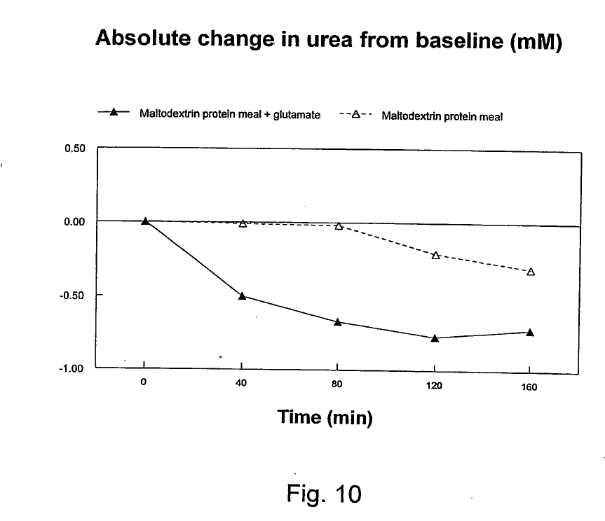

Also when a carbohydrate (maltodextrin) protein meal was added to the glutamate in young subjects, plasma urea decreased immediately after GLU intake (FIG. 10). This indicates that the nitrogen preservation is present both when GLU is ingested as a single amino acid or when combined with a carbohydrate protein meal.

Leucine Turnover

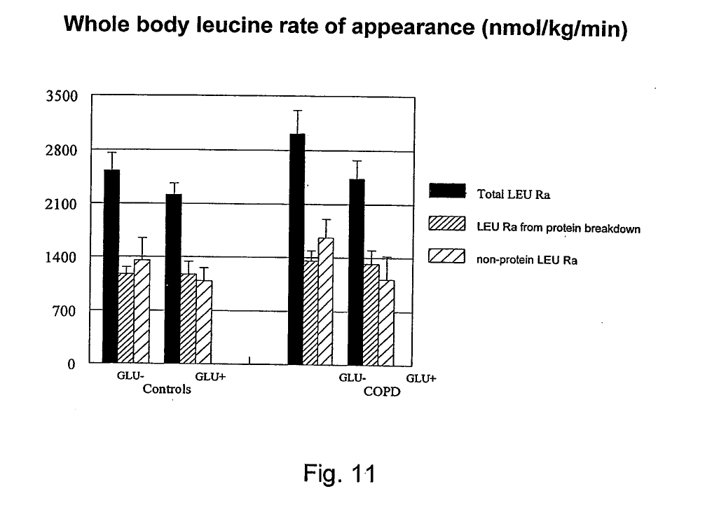

In the postabsorptive state, whole body leucine turnover measured by leucine tracer was increased at rest in the COPD patients compared to the healthy age-matched controls. Glutamate ingestion induced a reduction in whole body leucine turnover not related to protein turnover in both COPD and control subjects (FIG. 11).

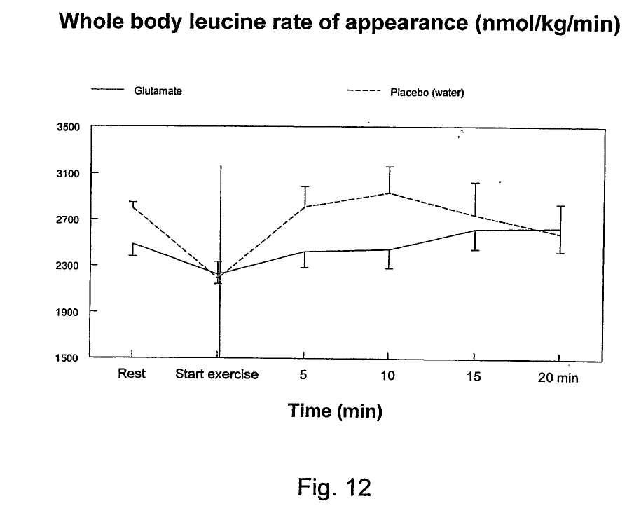

Glutamate ingestion also diminished the increase in whole body leucine turnover during cycle exercise in both COPD and control groups (FIG. 12). This was also due to a diminished increase in non-protein leucine turnover. These data indicate that glutamate reduces non-protein leucine turnover at rest and diminishes the exercise induced increase in leucine oxidation in COPD patients and healthy subjects.

Lactate Response to exercise

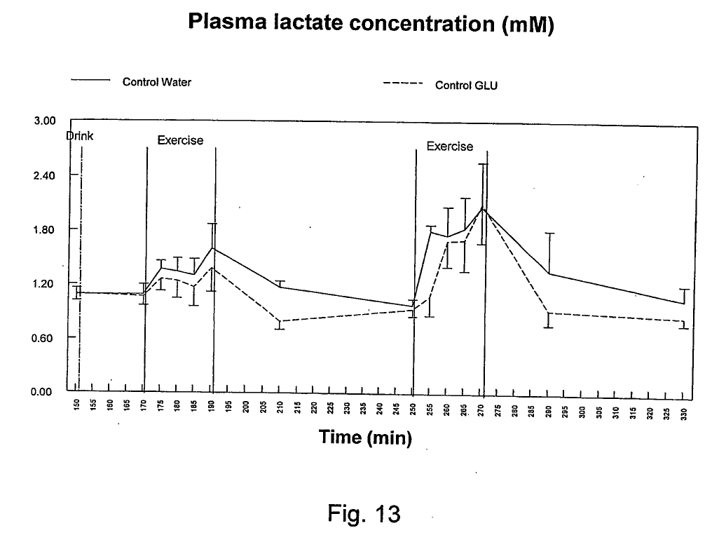

Glutamate ingestion resulted in a lower increase in plasma lactate concentration in the COPD group during exercise than when ingesting water (FIG. 13). This suggests that glutamate ingestion decreases the lactate response to exercise and in this way may reduce/delay the occurrence of muscle fatigue in this group of patients.

Side Effects

In the experiments the intake of glutamate is usually in the order of 6-7 g/hour for a period of 4-6 hours meaning a total intake of at least 30 grams. This intake is thus 30 times more than the estimated daily intake of MSG. However, none of the COPD patients or healthy age-matched control subjects indicated side effects (including symptoms according to the Chinese Restaurant syndrome) during or after GLU ingestion.

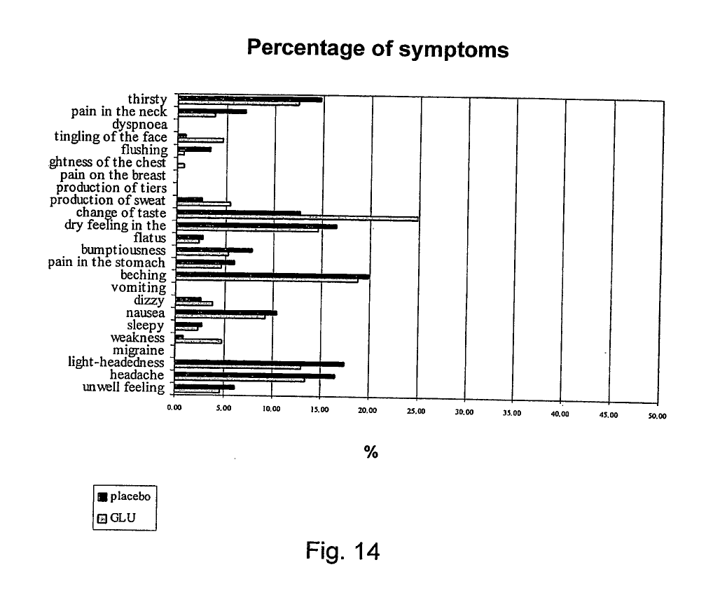

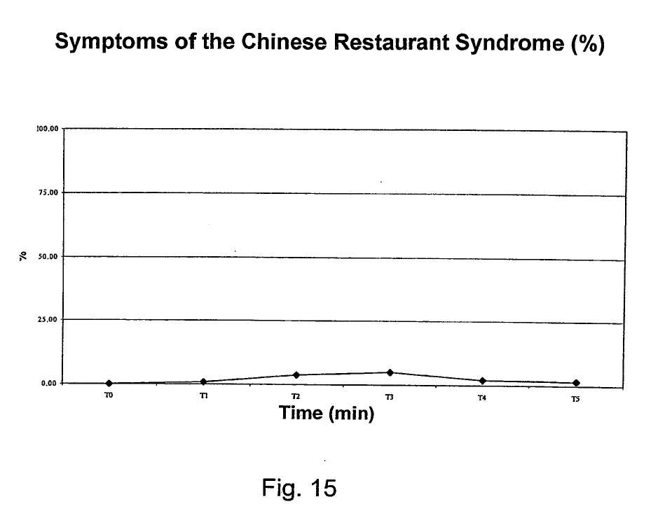

We confirmed this observation in a study in 26 healthy volunteers in which the primary focus was to more extensively elucidate whether and to what extent GLU ingestion as used in the above-mentioned protocol is inducing any symptoms including those of the CRS. This group of subjects ingested 30 mg GLU/kg body weight each 20 min and filled in a food tolerance questionnaire until 2 hours after the last ingestion. Moreover, a placebo (glutamine) was used for comparison. No differences were found in the number of complaints and the severity of the complaints between GLU and placebo ingestion (FIG. 14). Furthermore, less than 5% of the subjects were having the symptoms known as Chinese restaurant syndrome (FIG. 15). No significant differences were found with respect to these CRS effects between GLU and placebo. These data suggest that in the present invention GLU was associated with surprisingly better results with respect to side effects than previously observed with MSG.

### REFERENCES

1. van den Boom G, van Schayck C P, van Mollen M P, Tirimanna P R, den Otter J J, van Grunsven P M, et al., Am. J. Respir. Crit. Care Med. 1998; 158:1730-8.
2. Rutten-van Molken M P, Postma M J, Joore M A, Van Genugten M L, Leidl R, Jager J C. Respir. Med. 1999; 93:779-87.
3. American Thoracic Society. Am. Rev. Respir. Dis. 1995; 152:s77-s120.
4. Thurlbeck W M. Clin. Chest Med. 1990; 11:389-403.
5. American Thoracic Society/European Respiratory Society. Am. J. Respir. Crit. Care Med. 1999; 159:S1-40.
6. Hamilton A L, Killian K J, Summers E, Jones N L. Am. J. Respir. Crit. Care Med. 1995; 152: 2021-2031.
7. Gosselink R, Troosters T, Decramer M. Am. J. Respir. Crit. Care Med. 1996; 153:976-980.
8. Engelen M P K J, Schols A M W J, Does J D, Wouters E F M. Am. J. Clin. Nutr. 2000; 71:733-738.
9. Casaburi R, Patessio A, Ioli F, Zanaboni S, Donner C F, Wasserman K. Am. Rev. Respir. Dis. 1991; 143:9-18.
10. Sue D Y, Wasserman K, Moricca R B, Casaburi R. Chest 1988; 94:931-938.
11. Maltais F, Simard M, Simard C, Jobin J, Desgagnes P, LeBlanc P. Am. J. Respir. Crit. Care Med. 1996; 153:288-293.
12. Satta A, Migliori G B, Spanevello A, Neri M, Bottinelli R, Canepari M, et al., Eur. Respir. J. 1997; 10:2853-2860.
13. Jakobsson P, Jorfeldt L, Brundin A. Eur. Respir. J. 1990; 3:192-196.
14. Whittom F, Jobin J, Simard P M, Leblanc P, Simard C, Bernard S, et al. Med. Sci. Sports Exerc. 1998; 30:1467-1474.
15. Jakobsson P, Jorfeldt L. Respir. Med. 1995; 89:471-476.
16. Tada H, Kato H, Misawa T, Sasaki S, Hayashi S, Takahashi H, et al., Eur. Respir. J. 1992; 5:163-169.
17. Wuyam B, Payen J F, Levy P, Bensaidane H, Reutenauer H, Le Bas J F, et al., Eur. Respir. J. 1992; 5:157-162.
18. Pouw E M, Schols A M W J, Vusse vd G J, Wouters E F M. Am. J. Respir. Crit. Care Med. 1998; 157:453-457.
19. Pouw E M, Schols A M W J, Deutz N E P, Wouters E F M. Am. J. Respir. Crit. Care Med. 1998; 158:797-801.
20. Engelen M P K J, Schols A M W J, Does J D, Deutz N E P, Wouters E F M. Am. J. Respir. Crit. Care Med. 2000; 161:98-103.
21. Engelen M P K J, Wouters E F M, Deutz N E P, Does J D, Schols A M W J. Am. J. Respir. Crit. Care Med. 2001; 163:859-64.
22. Soguel Schenkel N, Burdet L, de Murait B, Fitting J W. Eur. Respir. J. 1996; 9:2584-2589.
23. Schols A M W J, Mostert R, Cobben N, Soeters P B, Wouters E F M. Chest 1991; 100:1287-1292.
24. Owens G R, Rogers R M, Pennock B E, Levin D. N. Engl. J. Med. 1984; 310:1218-1221.
25. Ferrari R, Ceconi C, Curello S, Alfieri O, Visioli O. Eur. Heart J. 1993; 14:25-30.
26. Wiesner R J, Deussen A, Borst M, Schrader J, Grieshaber M K. J. Mol. Cell. Cardiol. 1989; 21:49-59.
27. Hall van G, Saltin B, Vusse van der G J, Soderlund K, Wagenmakers A J M. J. Physiol. 1995; 489:251-261.
28. Hall van G, MacLean D A, Saltin B, Wagenmakers A J M. J. Physiol. 1996; 494:899-905.
29. Engelen M P K J, Schols A M W J, Does J D, Gosker H R, Deutz N E P, Wouters E F M. Am. J. Respir. Crit. Care Med. 2000; 162:1697-1704.
30. Mourtzakis, M, Graham T E. J. Appl. Physiol. 2003; 93:1251-9
31. Morrison W L, Gibson J N A, Scrimgeour C, Rennie M J. Clin. Sci. 1988; 75:415-420.
32. Schols A M W J, Deutz N E P, Mostert R, Wouters E F M. Monaldi Arch. Chest Med. 1993; 48:546-548.
33. Hofford J M, Milakofsky L, Vogel W H, Sacher R S, Savage G J, Pell S. Am. Rev. Respir. Dis. 1990; 141:902-908.
34. Engelen M P K J, Wouters E F M, Deutz N E P, Menheere P P, Schols A M W J. Am. J. Clin. Nutr. 2000; 72:1480-7.
35. Geha R S et al., J. Nutr. 2000; 130:1058S-62S.
36. Schaumburg H H, et al., Science 1969; 163:826-8.
37. Walker R, Lupien J R. J. Nutr. 2000; 130:1049S-52S.
38. Stegink L D, Filer L J, Baker G L. Am. J. Clin. Nutr. 1982; 36:1145-52.
39. Peng Y, et al., J. Nutr. 1973; 103:608-17.
40. Byck R, Schaumburg H H. N. Engl. J. Med. 1969; 281:275.
41. Graham T E, Sgro V, Friars D, Gibala M J. Am. J. Physiol. 2000; 278:E83-E89.
42. Milewski P J, Threlfall C J, Heath D F, Holbrook I B, Wilford K, Irving M H. Clin. Sci. 1982; 62:83-91
43. Roth E, Muhlbacher F, Rauhs R, Huk I, Soderland K, Funovics J. J.P.E.N. 1982; 6:240-4.
44. Roth E, Zoch G, Schultz F. Clin. Chem. 1985; 31:1305-9
45. Essen P, Wernerman J, Sonnenfeld T, Thunell S. Vinnars E. Clin. Physiol. 1992; 12:163-177.
46. Askanazi J, Furst P, Michelsen C B, Elwyn D H, Vinnars E, Gump F E, Stinchfield F E, Kinney J M. Ann. Surg. 1980; 191:465-72.
47. Henriksson J. J. Exp. Biol. 1991; 160: 149-165.
48. Matthews D E, Marano M A, Campbell R G. Am. J. Physiol. 1993; 264:E848-54.
49. Reeds P J, Burrin D G, Stoll B, Jahoor F. J. Nutr. 2000; 130:978S-82S.
50. Stegink L D, Filer L J, Jr., Baker G L. Am. J. Clin. Nutr. 1985; 42:220-5.
51. Ghezzi P, Bianchi M, Gianera L, Salmona M, Garattini S. Food Chem. Toxicol. 1985; 23: 975-8.

1. A composition suitable for the treatment or prophylaxis of COPD and other acute or chronic diseases in a mammal, especially a human being, comprising at least one of glutamate, other than mono sodium glutamate, and a precursor of glutamate selected from the group consisting of leucine, valine, isoleucine, and a keto acid thereof, in a daily dose for said mammal of at least 6 grams of the total of said glutamate and precursor forms thereof.

2. A composition as claimed in claim 1 comprising at least one of glutamate, other than mono sodium glutamate, and a precursor of glutamate selected from the group consisting of leucine, valine, isoleucine, and a keto acid thereof, in a daily dose for said mammal in a range of between 9 and 20 grams of the total of said glutamate and precursor forms thereof.

3. A composition as claimed in claim 1 which is a dietary food supplement where the amount of said glutamate or said precursor form thereof is subdivided in dosages of up to 3 grams, for regular administration to achieve continuously increasing glutamate level.

4. A composition as claimed in claim 1 which is a pharmaceutical composition where the amount of said glutamate or said precursor form thereof is subdivided in unit dosages of up to 3 grams, for regular administration to achieve continuously increasing glutamate level, the pharmaceutical composition further comprising a pharmaceutical acceptable carrier.

5. A method of preparing a unit dose medicament, comprising providing at least one of glutamate, other than mono sodium glutamate, and a precursor of glutamate selected from the group consisting of leucine, valine, isoleucine, and a keto acid thereof, in the medicament for treatment or prophylaxis of COPD and other acute or chronic diseases in a mammal, especially a human being, wherein the medicament is formulated to achieve a daily dose of at least 6 grams of the active ingredient of the medicament.

6. A method as claimed in claim 5, wherein the medicament is formulated in a unit dose form to achieve a daily dose in a range of between 9 and 20 grams of the active ingredient of the medicament.

7. A pharmaceutical composition as claimed in claim 4, which is formulated for oral or parenteral administration.

8. A pharmaceutical composition as claimed in claim 4, which is formulated to achieve a continuously increasing glutamate level.

9. A method as claimed in claim 5, wherein the medicament additionally contains one or more substances selected from the group of stimulants, hormones, analogues of such hormones, phyto-hormones, analogues of such phyto-hormones, and anti-oxidants.

10. A method of preventing or treating COPD and other acute or chronic diseases in a mammal, in particular a human, which comprises administering to said mammal a therapeutically effective amount of at least one of glutamate, other than mono sodium glutamate, and a precursor of glutamate selected from the group consisting of leucine, valine, isoleucine, and a keto acid thereof.

11. A method of preparing a food supplement or a prophylactic composition, comprising providing at least one of glutamate, other than mono sodium glutamate, and a precursor of glutamate selected from the group consisting of leucine, valine, isoleucine, and a keto acid thereof, in the food supplement or prophylactic composition to restore or increase the glutamate level in the body, in particular the muscles, of an individual.
